(19) United States
(12) Patent Application Publication
(10) Pub. No.: US 2008/0086082 A1
(43) Pub. Date: Apr. 10, 2008

(54) RADIOPAQUE MARKING TO DETECT BALLOON DEFLATION

(76) Inventor: Jeffrey S. BROOKS, Ra'anana (IL)

Correspondence Address: ABELMAN, FRAYNE & SCHWAB, 666 THIRD AVENUE, 10TH FLOOR, NEW YORK, NY, 10017, US

(21) Appl. No.: 11/868,631

(22) Filed: Oct. 8, 2007

Related U.S. Application Data
(60) Provisional application No. 60/850,071, filed on Oct. 6, 2006.

(51) Int. Cl. A61M 25/10

(52) U.S. Cl. 604/100.03

(57) ABSTRACT

Apparatus is provided, including a balloon configured for insertion into a body of a patient; and at least one marking coupled to the balloon, a state of the marking being indicative of a state of inflation of the balloon and detectable from outside of the body.

### CROSS-REFERENCES TO RELATED APPLICATIONS

The present application claims priority from U.S. Provisional Patent Application 60/850,071, entitled, “Radiopaque marking to detect balloon deflation,” filed Oct. 6, 2006, which is incorporated herein by reference.

### FIELD OF THE INVENTION

The present invention generally relates to implantable medical apparatus. Specifically, the present invention relates to assessing a state of apparatus implanted in the gastrointestinal tract.

### BACKGROUND OF THE INVENTION

Morbid obesity remains an ever-growing problem in the U.S. and throughout the world. Varying treatments have been developed, including gastric bypass surgery, gastric banding, and implanted gastric balloons.

U.S. Pat. No. 6,981,980 to Sampson, et al., which is incorporated herein by reference, describes a self-inflating, intragastric balloon device activated by addition of an activating liquid into an acid or water soluble vessel located within the balloon. The device may be provided to medical personnel as part of a kit that further contains a syringe and the activating liquid. The device also can have acid or pepsin degradable portions that cause it to deflate after residing in the stomach for a prolonged period of time during which it imparts a feeling of satiety in the patient. After deflation, the device can pass through the pylorus and the rest of the digestive system without injury. The '980 patent describes a bladder that is made of any substantially liquid-impermeable material. The material may be non-elastic or semi-elastic, such as Dacron®, Nylon® and the like, with Nylon® being preferred. Alternatively, the material may be highly elastic, such as rubber, latex and the like. Further, the bladder may have a mono-layer, bi-layer or multi-layer construction. For instance, the bladder may have an inner layer of Nylon® or ethyl vinyl acetate and an outer layer of silicone for better biocompatibility. In addition, the substantially liquid-impermeable material could contain a radiopaque substance to enable visualization of the balloon in the patient's stomach. Alternatively, the balloon could have a band of radiopaque material, like a metal foil, around its circumference to enable visualization.

U.S. Pat. No. 7,037,344 to Kagan, et al., which is incorporated herein by reference, describes an apparatus and method for treatment of morbid obesity including a system of components for effectively reducing stomach volume, bypassing a portion of the stomach and/or small intestines, reducing nutrient absorption in the stomach and/or small intestines and/or depositing minimally or undigested food farther than normal into the intestines, thereby stimulating intestinal responses. The components described include an artificial stoma device, a gastric sleeve device, an intestinal sleeve device and a combined gastrointestinal sleeve device. The wall of the sleeve may be reinforced with rings or a spiral made of wire and/or plastic. Optionally, these components can include radiopaque materials for visualization of the device when it is in the body. Each component of the system includes one or more radiopaque and/or sonoreflective markers for enhanced imaging by X-ray, fluoroscopy and/or ultrasonic imaging.

The following article, which is incorporated herein by reference, may be of interest:

Doldi SB et al., “Intragastric balloon: Another option for treatment of obesity and morbid obesity,” Hepatogastroenterology. 2004 January-February; 51(55):294-7

### SUMMARY OF THE INVENTION

In some embodiments of the present invention, a radiopaque marking is coupled to a balloon that is designated for insertion into the gastrointestinal tract of a patient. Deformations of the radiopaque marking correspond to changes in the state of inflation of the balloon, and are detectable from outside of the body of the patient, typically by fluoroscopy. If deformations of the marking are indicative of deflation of the balloon, the balloon is typically re-inflated or replaced with a new balloon.

The radiopaque marking typically comprises one or more lines, e.g., continuous lines (uninterrupted by gaps), or dashed lines (interspersed with gaps). Alternatively or additionally, the radiopaque marking comprises a plurality of spots, typically arranged in lines or in a geometric pattern (for example, a grid). Further alternatively or additionally, the radiopaque marking covers all or a substantial portion of the entire balloon.

There is therefore provided, in accordance with an embodiment of the present invention, apparatus for use in a gastrointestinal tract of a subject, the apparatus including:

a balloon configured for insertion into a body of a patient; and

at least one marking coupled to the balloon, a state of the marking being indicative of a state of inflation of the balloon and detectable from outside of the body.

In an embodiment, the at least one marking is configured to assume a straight configuration thereof at least one level of inflation of the balloon.

In an embodiment, the at least one marking is configured to assume a wavy configuration thereof at least one level of inflation of the balloon.

In an embodiment, the at least one marking includes one or more lines.

In an embodiment, the one or more lines include two or more lines that are arranged in a grid.

In an embodiment, the one or more lines include two or more parallel lines.

In an embodiment, the one or more lines include two or more non-parallel lines.

In an embodiment, the at least one marking includes a plurality of spots.

In an embodiment, the plurality of spots are arranged in a geometrical pattern.

In an embodiment, the marking includes a radiopaque marking.

There is additionally provided, in accordance with an embodiment of the present invention, a method including:

placing a balloon into a body of the patient during a medical procedure;

following the medical procedure, observing a state of at least one marking coupled to the balloon; and

determining whether the balloon is at least partially deflated, in response to the state of the at least one marking.

In an embodiment, determining whether the balloon is at least partially deflated includes identifying a wavy configuration of the at least one marking.

In an embodiment, observing the state of the at least one marking includes determining whether the marking is straight, and includes determining that the balloon is sufficiently inflated at least in part in response to the marking being straight.

In an embodiment, observing includes fluoroscopically observing.

The present invention will be more fully understood from the following detailed description of embodiments thereof, taken together with the drawings, in which:

### BRIEF DESCRIPTION OF THE DRAWINGS

FIG. 1 is a schematic illustration of a radiopaque marking coupled to a gastric balloon in its inflated state, in accordance with an embodiment of the present invention;

FIG. 2 is a schematic illustration of the gastric balloon of FIG. 1 in its deflated state, in accordance with an embodiment of the present invention;

FIG. 3 is a schematic illustration of another radiopaque marking coupled to the gastric balloon of FIG. 1, in accordance with an embodiment of the present invention;

FIG. 4 is a schematic illustration of the gastric balloon of FIG. 3 in its deflated state, in accordance with an embodiment of the present invention;

FIG. 5 is a schematic illustration of a radiopaque marking coupled to a gastric balloon in its inflated state, in accordance with an embodiment of the present invention; and

FIG. 6 is a schematic illustration of the gastric balloon of FIG. 5 in its deflated state, in accordance with an embodiment of the present invention.

### DETAILED DESCRIPTION OF EMBODIMENTS

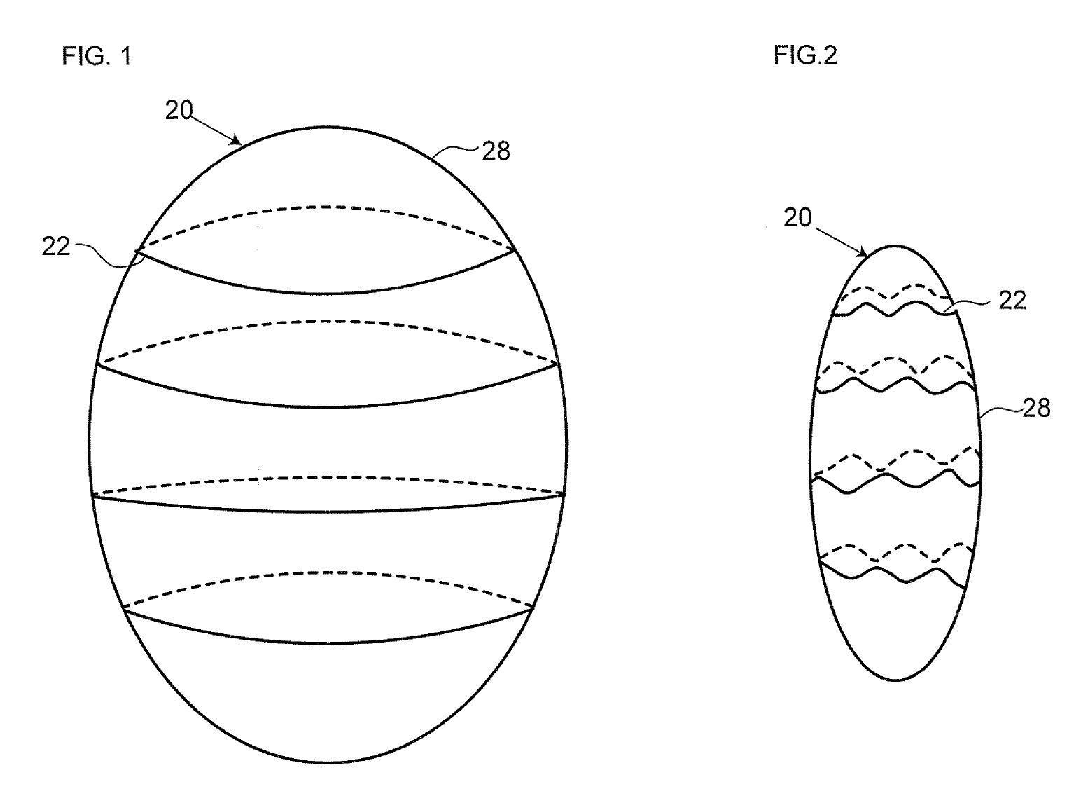

Reference is now made to FIGS. 1 and 2, which are schematic illustrations of apparatus 20 comprising radiopaque markings 22 coupled to a gastric balloon 28, in accordance with an embodiment of the present invention. FIG. 1 shows gastric balloon 28 in its inflated state, and FIG. 2 shows gastric balloon 28 in a partially deflated state. Changes in the shape of radiopaque markings 22 typically provide a physician with the ability to fluoroscopically detect partial or complete deflation of the balloon once implanted in the gastrointestinal tract. Although devices and methods are described in some embodiments as being useful for detecting deflation of the balloon in the stomach, it is to be understood that the methods and devices described herein can be used with a balloon placed at other sites within a patient's body, as well. Similarly, the scope of the present invention includes using a radiopaque marking or other marking to facilitate detection of a change of shape of other implanted medical apparatus.

As shown in FIG. 1, radiopaque markings 22 are arranged as lines of latitude upon balloon 28 in straight, continuous lines. Given the inflated condition of the balloon, the markings assume a straight configuration, indicating under fluoroscopy that the balloon is inflated.

Typically, but not necessarily, markings 22 are disposed on the inner surface of balloon 28.

FIG. 2 shows gastric balloon 28 in a deflated state, causing radiopaque markings 22 to form wavy lines due to the deflated state of the balloon.

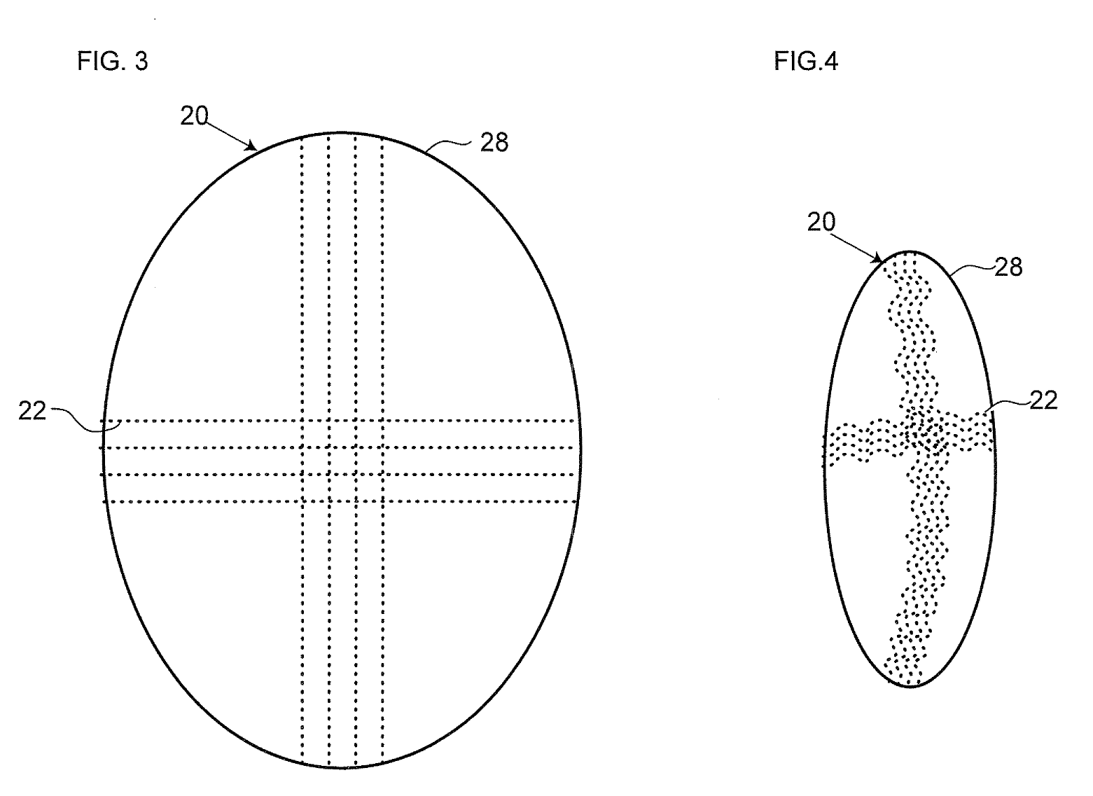

FIGS. 3 and 4 illustrate a configuration of radiopaque markings 22 coupled to gastric balloon 28 to form a geometrical pattern, such as a grid, in accordance with an embodiment of the present invention. In some embodiments of the present invention, the markings comprise a plurality of spots, as illustrated in FIGS. 3 and 4.

As shown in FIG. 3, balloon 28 is in an inflated state. Radiopaque markings 22 are aligned along latitude and longitude lines of balloon 28, and are generally straight due to the distention of the inflated balloon. FIG. 4 correspondingly represents a deflated state of gastric balloon 28, in which radiopaque markings 22 form wavy lines due to the shrinking of the balloon.

It is to be appreciated that other aspects of radiopaque marking 22 may change, in addition to or instead of the straight versus wavy shape of the markings. For example, the distance between different aspects of the radiopaque markings (e.g., lines or spots) decreases in response to deflation of gastric balloon 28.

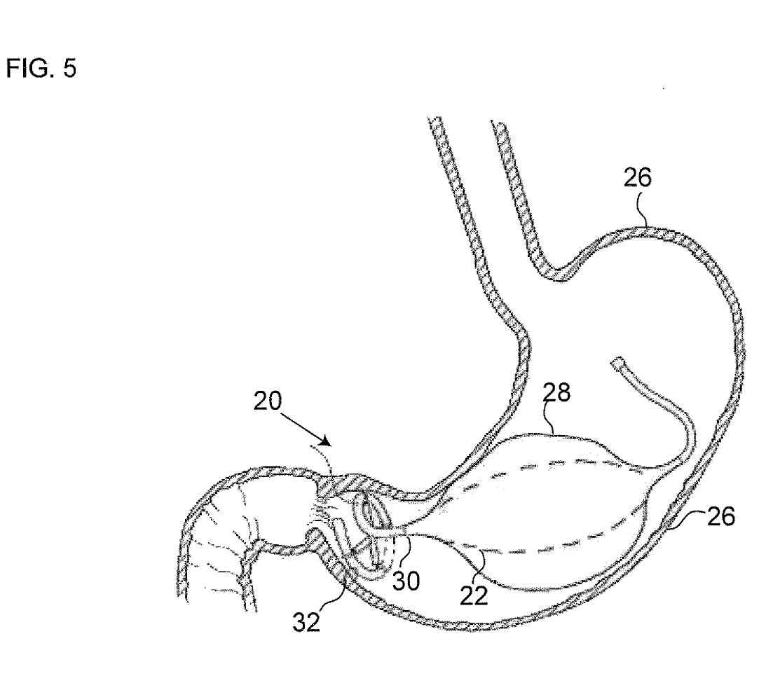

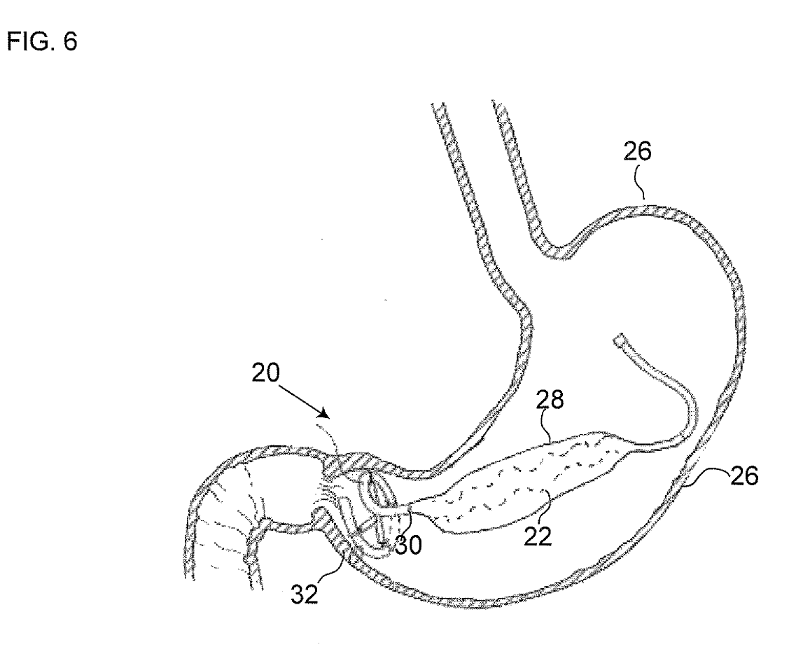

Reference is now made to FIGS. 5 and 6, which are schematic illustrations of gastric balloon 28 in the stomach 26 of the patient, in respective inflated and deflated states, in accordance with an embodiment of the present invention. Gastric balloon 28 is oriented within stomach 26 such that an anchor 30 coupled to the distal end of gastric balloon 28 is disposed within the antrum 32. In the embodiment shown, radiopaque markings 22 comprise dashed lines interspersed with short gaps, and function generally as described hereinabove to indicate the level of inflation of gastric balloon 28.

The scope of the present invention includes the use of configurations of the radiopaque marking other than those described hereinabove and shown in the figures. For example, the radiopaque marking may include various combinations of lines or patterns, for example grids and/or parallel or non-parallel lines, so as to occupy part of the balloon or substantially the entire balloon. For some applications, the entire balloon is radiopaque, and the size of the balloon is used as a direct indicator of the level of inflation of the balloon.

Embodiments described herein may be practiced in combination with techniques described in one or more of the following applications, which are assigned to the assignee of the present patent application and are incorporated herein by reference:

U.S. patent application Ser. No. 11/132,855, filed May 18, 2005;

U.S. Provisional Patent Application 60/639,843, filed Dec. 27, 2004;

PCT Patent Application PCT/IL2005/001381, filed Dec. 27, 2005;

U.S. Provisional Patent Application 60/787,124 filed by Brooks on Mar. 28, 2006, entitled, “Floating gastrointestinal anchor,” and

a US provisional patent application filed by Brooks on Jun. 20, 2006, entitled, “Floating gastrointestinal anchor.”

The scope of the present invention includes embodiments described in each of these applications.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
a balloon configured for insertion into a body of a patient; and
at least one marking coupled to the balloon, a state of the marking being indicative of a state of inflation of the balloon and detectable from outside of the body.

2. The apparatus according to claim 1, wherein the at least one marking is configured to assume a straight configuration thereof at least one level of inflation of the balloon.

3. The apparatus according to claim 1, wherein the at least one marking is configured to assume a wavy configuration thereof at least one level of inflation of the balloon.

4. The apparatus according to claim 1, wherein the at least one marking comprises one or more lines.

5. The apparatus according to claim 4, wherein the one or more lines comprise two or more lines that are arranged in a grid.

6. The apparatus according to claim 4, wherein the one or more lines comprise two or more parallel lines.

7. The apparatus according to claim 4, wherein the one or more lines comprise two or more non-parallel lines.

8. The apparatus according to claim 1, wherein the at least one marking comprises a plurality of spots.

9. The apparatus according to claim 8, wherein the plurality of spots are arranged in a geometrical pattern.

10. The apparatus according to claim 1, wherein the marking comprises a radiopaque marking.

11. A method, comprising:
placing a balloon into a body of the patient during a medical procedure;
following the medical procedure, observing a state of at least one marking coupled to the balloon; and
determining whether the balloon is at least partially deflated, in response to the state of the at least one marking.

12. The method according to claim 11, wherein determining whether the balloon is at least partially deflated comprises identifying a wavy configuration of the at least one marking.

13. The method according to claim 11, wherein observing the state of the at least one marking comprises determining whether the marking is straight, and wherein the method comprises determining that the balloon is sufficiently inflated at least in part in response to the marking being straight.

14. The method according to claim 11, wherein observing comprises fluoroscopically observing.
